(19) United States
(12) Patent Application Publication
(10) Pub. No.: US 2023/0218297 A1
(43) Pub. Date: Jul. 13, 2023

(54) Reversible Vasectomy Device

(71) Applicants: Mohammed Muddassir Mohiuddin, Frederick, MD (US); Aabid Mohammed Mohiuddin, Frederick, MD (US)

(72) Inventors: Mohammed Muddassir Mohiuddin, Frederick, MD (US); Aabid Mohammed Mohiuddin, Frederick, MD (US)

(21) Appl. No.: 17/572,570

(22) Filed: Jan. 10, 2022

(51) Int. Cl. A61F 6/20; A61F 2/94

(52) U.S. Cl. CPC A61F 6/20; A61F 2/94; A61F 2210/0004; A61F 2220/0016

(57) ABSTRACT

A reversible vasectomy device suitable for reverse vasectomy operations is presented. More specifically, the present invention may take the form of a stent device that installs along the severed vas deferens such that the vas deferens ends are opened and coupled together. The reversible vasectomy device is made out of an absorbable material, allowing the reversible vasectomy device to be absorbed by the body, leaving only the connected vas deferens once the healing process is completed. The reversible vasectomy device contains a first connection element, a second connection element, and a mid-section. The mid-section is connected between the first connection element and the second connection element. The first connection element, the second connection element, and the mid-section are in fluid communication with each other.

### FIELD OF THE INVENTION

The field of the present invention relates generally to birth control devices. More specifically, devices for providing a reversible vasectomy.

### BACKGROUND OF THE INVENTION

A vasectomy or vas occlusion is a procedure by which the vas deferens of a male are blocked or severed in order to prevent the passage of sperm through the vas deferens. The vas deferens extends from the epididymis, at the testicles, to the prostate where it normally delivers sperm to the ejaculatory ducts. Severing or blocking the vas deferens provides sterility. While vasectomies are an exceedingly common form of male birth control, the procedure is generally considered permanent. A significant number of men who have had a vasectomy, subsequently wish to reverse the procedure to regain fertility. There are a variety of approaches to vasectomy reversal but all tend to be difficult, costly, and are not always successful.

Therefore, it is an objective of the present invention to provide a device suitable for a reverse vasectomy. The present invention is easy to install, allowing a surgeon to connect the severed vas deferens together without any use of special tools or complicated connection implements. The present invention further features absorbable and anti-inflammatory characteristics, such that the present invention is absorbed within the body while aiding in the healing process of the connected ends of the vas deferens.

### SUMMARY OF THE INVENTION

The present invention is a reversible vasectomy device suitable for reverse vasectomy operations. More specifically, the present invention may take the form of a stent device that installs along the severed vas deferens such that the vas deferens ends are opened and coupled together. In the preferred embodiment of the present invention, the reversible vasectomy device is made out of an absorbable material, allowing the reversible vasectomy device to be absorbed by the body, leaving only the connected vas deferens once the healing process is completed. In the preferred embodiment of the present invention, the reversible vasectomy device is made out of an anti-inflammatory material. The reversible vasectomy device aids in healing of the connected vas deferens by being made out of an anti-inflammatory material. In the preferred embodiment of the present invention, the reversible vasectomy device is made out of cortisone, but may be made out of any other suitable material. More specifically, the reversible vasectomy device may take the form of a stent-like structure made out of compressed cortisone powder with added binding agents that are also absorbable. The reversible vasectomy device comprises a first connection element, a second connection element, and a mid-section. The mid-section is connected between the first connection element and the second connection element. The first connection element, the second connection element, and the mid-section are in fluid communication with each other.

### BRIEF DESCRIPTION OF THE DRAWINGS

FIG. 1 is a top perspective view of the present invention.

FIG. 2 is a side view of the present invention.

FIG. 3 is a cross sectional view of the present invention taken along cutting lines A-A in FIG. 2.

FIG. 4 is a detailed view of the present invention taken along circle B in FIG. 3.

FIG. 5 is a detailed view of the present invention taken along circle C in FIG. 3.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

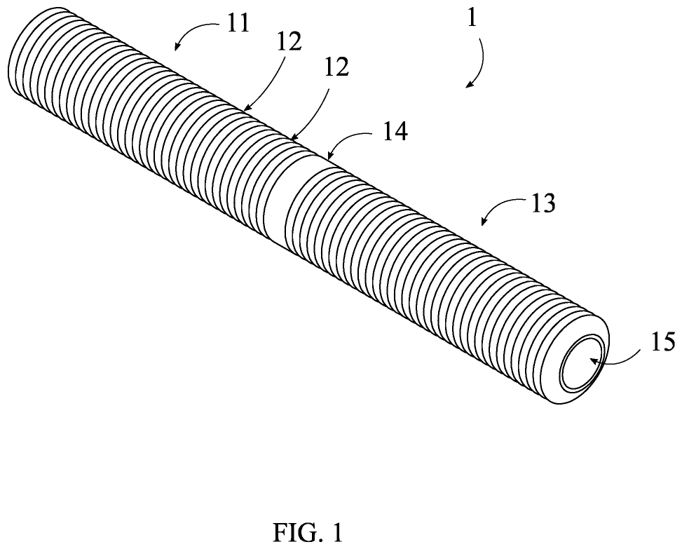

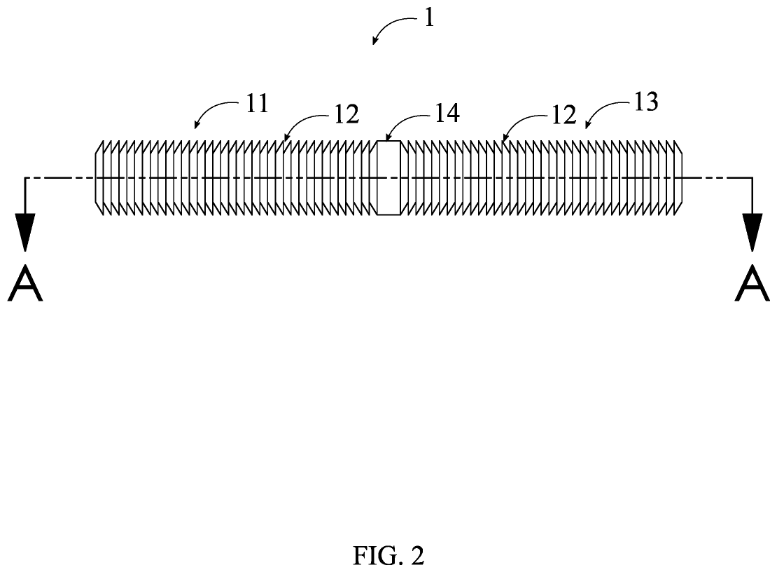

In reference to FIGS. 1-3, the present invention is a reversible vasectomy device 1 suitable for reverse vasectomy operations. More specifically, the present invention may take the form of a stent device that installs along the severed vas deferens such that the vas deferens ends are opened and coupled together. In the preferred embodiment of the present invention, the reversible vasectomy device 1 is made out of an absorbable material, allowing the reversible vasectomy device 1 to be absorbed by the body, leaving only the connected vas deferens once the healing process is completed. In the preferred embodiment of the present invention, the reversible vasectomy device 1 is made out of an anti-inflammatory material. The reversible vasectomy device 1 aids in healing of the connected vas deferens by being made out of an anti-inflammatory material. In the preferred embodiment of the present invention, the reversible vasectomy device 1 is made out of cortisone, but may be made out of any other suitable material. More specifically, the reversible vasectomy device 1 may take the form of a stent-like structure made out of compressed cortisone powder with added binding agents that are also absorbable. The reversible vasectomy device 1 comprises a first connection element 11, a second connection element 13, and a mid-section 14. The mid-section 14 is connected between the first connection element 11 and the second connection element 13. The first connection element 11, the second connection element 13, and the mid-section 14 are in fluid communication with each other.

In the preferred embodiment of the present invention, the first connection element 11 and the second connection element 13 serves as the connection portions of the reversible vasectomy device 1 that connects the two severed ends of the vas deferens together. In various embodiments, the first connection element 11 and the second connection element 13 may take the form of any suitable connection element that couples and opens the vas deferens tubes together, such as, but not limited to threaded connectors, collet style connectors, or any other suitable type of connection implement. In the preferred embodiment of the present invention, the mid-section 14 may take the form of a dividing body 141 that separates the first connection element 11 and the second connection element 13 from each other. This serves as a specified connection border for the first connection element 11 and the second connection element 13, where the vas deferens ends will meet along the mid-section 14 when the vas deferens is slipped along the first connection element 11 and the second connection element 13. The mid-section 14 serves as a guide to aid in fully installing the severed vas deferens ends along the first connection element 11 and the second connection element 13.

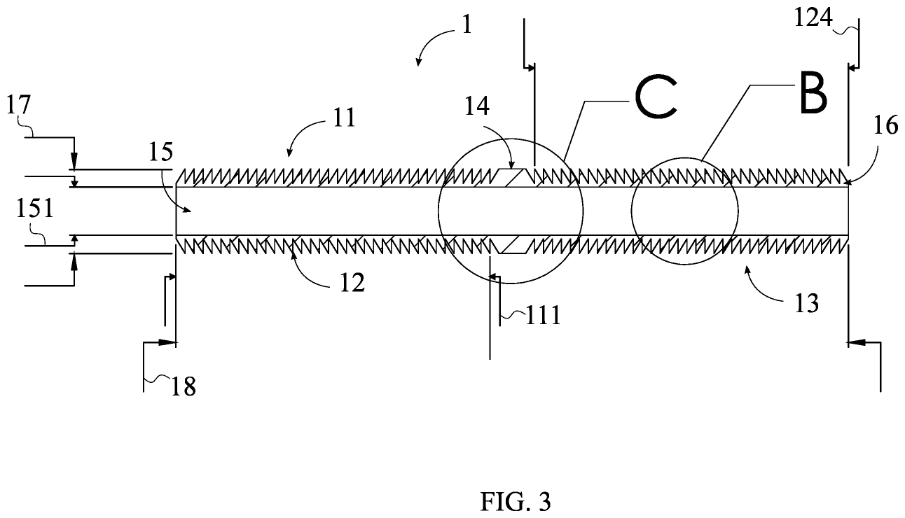

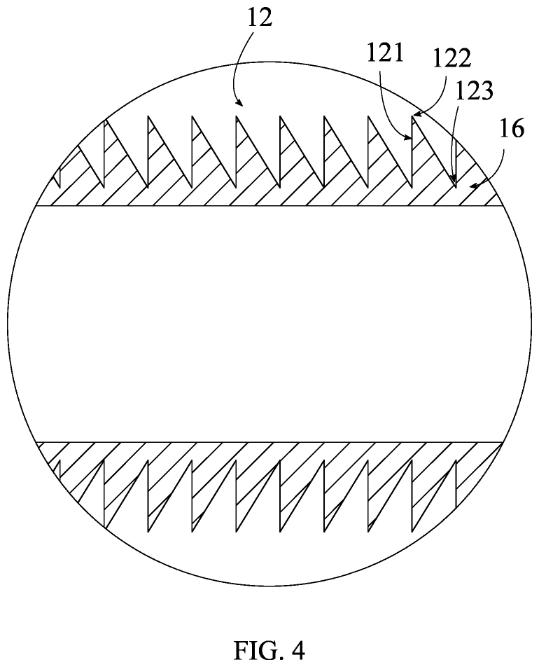

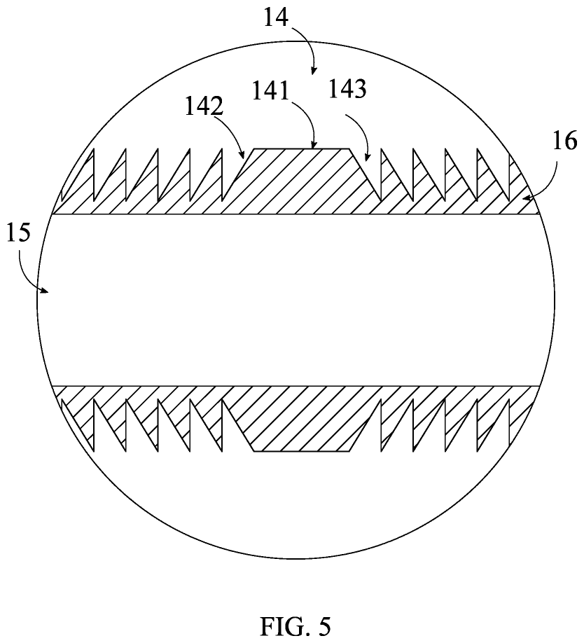

The first connection element 11 and the second connection element 13 comprises a plurality of barbs 12, as shown in FIGS. 1-5. The plurality of barbs 12 traverses along the first connection element 11 and the second connection element 13. Each of the plurality of barbs 12 comprises a barb body 121, a first engagement portion 122, and a second engagement portion 123, as shown in FIGS. 4-5. The first engagement portion 122 and the second engagement portion 123 are positioned terminally opposite to each other along the barb body 121. In the preferred embodiment of the present invention, the plurality of barbs 12 serves as the primary means of connecting the vas deferens ends to the first connection element 11 and the second connection element 13. The vas deferens ends will slip along the first connection element 11 and the second connection element 13, where the plurality of barbs 12 is configured to grasp and interlock the inner tube portion of the vas deferens such that they do not slip out of the first connection element 11 and the second connection element 13. In the preferred embodiment of the present invention, the plurality of barbs 12 comprises a cross-sectional profile 16 that is triangular in shape, as shown in FIGS. 3-5, but can take form of any other shape.

In reference to FIGS. 1 and 3-5, the reversible vasectomy device 1 further comprises a fluid channel 15. The fluid channel 15 traverses through the first connection element 11, the second connection element 13 and the mid-section 14. The fluid channel 15 may take the form of a channel opening within the reversible vasectomy device 1 that allows matured sperm to flow through the connected ends of the vas deferens through the reversible vasectomy device 1. In various embodiments, the fluid channel 15 is scaled to any suitable specified inner diameter.

In reference to FIG. 5, the mid-section 14 comprises a dividing body 141, a first end 142, and a second end 143. The first end 142 and the second end 143 are positioned terminally opposite to each other along the dividing body 141. The first connection element 11 is connected adjacent to the first end 142. The second connection element 13 is connected adjacent to the second end 143. In the preferred embodiment of the present invention, the first end 142 and the second end 143 serves as the installation end portions of the first connection element 11 and the second connection element 13, respectively, where the first end 142 and the second end 143 are configured to serve as installation guides where one vas deferens end installs along the first connection element 11 and stops at the first end 142 of the mid-section 14 and the other vas deference end installs along the second connection element 13 and stops at the second end 143 of the mid-section 14. The dividing body 141 spaces the connected vas deferens ends in length, such that the connected vas deferens ends are adequately spaced to heal properly along the reversible vasectomy device 1. In the preferred embodiment of the present invention, the dividing body 141 is scaled to a specified length.

In reference to FIG. 3, the reversible vasectomy device 1 further comprises a specified overall length 18 ranging from 3 to 5 centimeters. In the preferred embodiment, the first connection element 11 comprises a specified first connection length 111 ranging from 1.5 to 2.5 centimeters. In the preferred embodiment, the second connection element 12 comprises a specified second connection length 124 ranging from 1.5 to 2.5 centimeters. In the preferred embodiment, the fluid channel 14 comprises a specified fluid channel inner diameter 151 ranging from 1 to 2 French. In the preferred embodiment, the reversible vasectomy device 1 further comprises a specified outer diameter 17 ranging from 3 to 4 French.

Although the invention has been explained in relation to its preferred embodiment, it is to be understood that many other possible modifications and variations can be made without departing from the spirit and scope of the invention as hereinafter claimed.

What is claimed is:

1. A reversible vasectomy device comprising:
a first connection element;
a second connection element;
a mid-section;
a fluid channel;
the mid-section being connected between the first connection element and the second connection element;
the fluid channel traversing through the first connection element, the second connection element and the mid-section; and
the first connection element, the second connection element, the mid-section, and the fluid channel being in fluid communication with each other.

2. The reversible vasectomy device as claimed in claim 1, comprising:
the first connection element and the second connection element comprising a plurality of barbs; and
the plurality of barbs traversing along the first connection element and the second connection element.

3. The reversible vasectomy device as claimed in claim 2, comprising:
each of the plurality of barbs comprising a barb body, a first engagement portion, and a second engagement portion;
the first engagement portion and the second engagement portion being positioned terminally opposite to each other along the barb body;

4. The reversible vasectomy device as claimed in claim 1, comprising:
the mid-section comprising a dividing body, a first end, and a second end;
the first end and the second end being positioned terminally opposite to each other along the dividing body;
the first connection element being connected adjacent to the first end; and
the second connection element being connected adjacent to the second end.

5. The reversible vasectomy device as claimed in claim 1, wherein the reversible vasectomy device is made out of an absorbable material.

6. The reversible vasectomy device as claimed in claim 1, wherein the reversible vasectomy device is made out of an anti-inflammatory material.

7. The reversible vasectomy device as claimed in claim 1, wherein the reversible vasectomy device is made out of cortisone.

8. The reversible vasectomy device as claimed in claim 2, wherein the plurality of barbs comprises a cross-sectional profile that is triangular in shape.

9. The reversible vasectomy device as claimed in claim 1, wherein the reversible vasectomy device further comprises a specified overall length ranging from 3 to 5 centimeters.

10. The reversible vasectomy device as claimed in claim 1, wherein the first connection element comprises a specified first connection length ranging from 1.5 to 2.5 centimeters.

11. The reversible vasectomy device as claimed in claim 1, wherein the second connection element comprising a specified second connection length ranging from 1.5 to 2.5 centimeters.

12. The reversible vasectomy device as claimed in claim 1, wherein the fluid channel comprising a specified fluid channel inner diameter ranging from 1 to 2 French.

13. The reversible vasectomy device as claimed in claim 1, wherein the reversible vasectomy device further comprising a specified outer diameter ranging from 3 to 4 French.
